(19) United States
(12) Patent Application Publication
(10) Pub. No.: US 2019/0337932 A1
(43) Pub. Date: Nov. 7, 2019

(54) PROCESS FOR PREPARATION OF DACLATASVIR AND SALTS

(71) Applicant: Gienmark Pharmaceuticals Limited, Mumbai (IN)

(72) Inventors: Prem Chand, Navi Mumbai (IN); Yogesh YADAV, Meerut (IN); Amit Anant THANEDAR, Nashik (IN); Mohammad A. KHAN, Thane (IN); Ranjeet NAIR, Mumbai (IN); Sukumar SINHA, Navi Mumbai (IN); Shekhar Bhaskar BHiRUD, Mumbai (IN)

(21) Appl. No.: 16/312,359

(22) PCT Filed: Jul. 13, 2017
(86) PCT No.: PCT/IB2017/054230

§ 371 Date: Dec. 21, 2018

(30) Foreign Application Priority Data
Jul. 18, 2016 (IN) 201621024505

(51) Int. Cl. C07D 403/14; C07C 55/08

(52) U.S. Cl. CPC C07D 403/14; C07B 2200/13; C07C 55/08

(57) ABSTRACT

The present invention relates to crystalline daclatasvir dihydrochloride hydrate and process for its preparation.

### PRIORITY

This application claims the benefit of IN201621006730, filed on Jul. 18, 2016, the content of which is incorporated herein by reference.

### FIELD OF THE INVENTION

The present invention relates to crystalline daclatasvir dihydrochloride hydrate and process for its preparation.

### BACKGROUND OF THE INVENTION

Daclatasvir, also known as carbamic acid, N,N′-[[1,1′-biphenyl]-4,4′-diylbis[1H-imidazole-5,2-diyl-(2S)-2,1-pyrrolidinediyl[(1S)-1-(1-methylethyl)-2-oxo-2,1-ethanediyl]]]bis-C,C′-dimethyl ester, is represented by the structure of formula I.

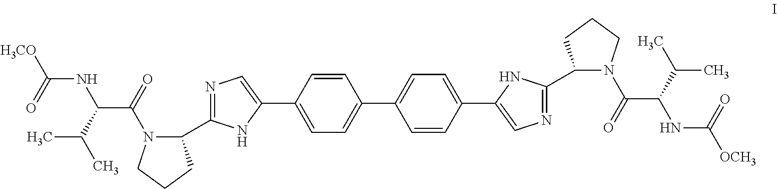 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]

Daclatasvir dihydrochloride, a compound of formula II, is a hepatitis C virus (HCV) NS5A inhibitor indicated for use with sofosbuvir, with or without ribavirin, for the treatment of chronic HCV genotype 1 or 3 infection.

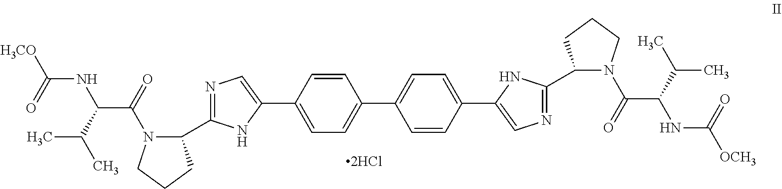 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]

The object of the present invention is to provide crystalline daclatasvir dihydrochloride hydrate.

### SUMMARY OF THE INVENTION

The present invention provides a crystalline daclatasvir dihydrochloride hydrate wherein the molar ratio of daclatasvir dihydrochloride and water is in the range of 1:0.5 to 1:6.

In another embodiment, the present invention provides a process for the preparation of crystalline daclatasvir dihydrochloride hydrate, the process comprising:
(a) providing daclatasvir in a solvent or a mixture of solvents;
(b) adding hydrochloric acid, optionally in the presence of a solvent, to the mixture obtained in step (a) to form daclatasvir dihydrochloride;
(c) optionally isolating daclatasvir dihydrochloride from the mixture of step (b);
(d) providing the daclatasvir dihydrochloride obtained in step (b) or step (c) in water to form a mixture;
(e) obtaining crystalline daclatasvir dihydrochloride hydrate from the mixture of step (d), and
(f) isolating the crystalline daclatasvir dihydrochloride hydrate.

In another embodiment, the present invention provides a process for the preparation of crystalline daclatasvir dihydrochloride hydrate, the process comprising: (a) dissolving daclatasvir dihydrochloride in water, optionally in the presence of a solvent, to form a solution; (b) heating the solution obtained in step (a) to a temperature of about 40° C. to 110° C.; (c) optionally maintaining the solution of step (b) at the temperature of about 40° C. to 110″C; (d) cooling the solution of step (b) or step (c) to a temperature of about −5° C. to 35° C.; and (e) isolating the crystalline daclatasvir dihydrochloride hydrate.

In another embodiment, the present invention provides a salt of daclatsavir with malonic acid.

In another embodiment, the present invention provides a process for preparation of daclatasvir dihydrochloride comprising
(a) reacting daclatasvir with malonic acid to form daclatasvir malonate;
(b) reacting daclatasvir malonate with a base to form daclatasvir;
(c) reacting the daclatasvir of step b with hydrochloric acid to form daclatasvir hydrochloride.

In another embodiment, the present invention provides use of daclatasvir malonate to prepare daclatasvir dihydrochloride or hydrate thereof.

In another embodiment, the present invention provides a process for daclatasvir dihydrochloride comprising
(a) preparing daclatasvir malonate; and
(b) converting daclatasvir malonate to daclatasvir hydrochloride.

### BRIEF DESCRIPTION OF THE DRAWINGS

FIG. 1 is a characteristic XRPD of crystalline daclatasvir dihydrochloride hydrate as obtained in Example 7a.

FIG. 2 is a TGA thermogram of crystalline daclatasvir dihydrochloride hydrate as obtained in Example 7a.

FIG. 3 is a DSC thermogram of crystalline daclatasvir dihydrochloride hydrate as obtained in Example 7a.

FIG. 4 is a TGA thermogram of crystalline daclatasvir dihydrochloride hydrate as obtained in Example 7b.

FIG. 5 is a DSC thermogram of crystalline daclatasvir dihydrochloride hydrate as obtained in Example 7b.

FIG. 6 is a TGA thermogram of crystalline daclatasvir dihydrochloride hydrate as obtained in Example 7c.

FIG. 7 is a DSC thermogram of crystalline daclatasvir dihydrochloride hydrate as obtained in Example 7c.

FIG. 8 is characteristic XRPD of crystalline daclatasvir malonate.

### DETAILED DESCRIPTION OF THE INVENTION

The present invention provides a crystalline daclatasvir dihydrochloride hydrate.

In the present application, the term “room temperature” means a temperature of about 25° C. to about 30° C.

In one embodiment, the present invention provides a crystalline daclatasvir dihydrochloride hydrate wherein the molar ratio of daclatasvir dihydrochloride and water is in the range of 1:0.5 to 1:6.

In one embodiment, the present invention provides a crystalline daclatasvir dihydrochloride hydrate wherein the molar ratio of daclatasvir dihydrochloride and water is in the range of 1:2 to 1:5.

In one embodiment, the present invention provides a crystalline daclatasvir dihydrochloride hydrate, wherein the molar ratio of daclatasvir dihydrochloride and water is in the range of 1:3 to 1:5.

In one embodiment, the present invention provides a crystalline daclatasvir dihydrochloride hydrate characterized by an X-ray powder diffraction (XRPD) spectrum having peak reflections at about 9.5, 11.0, 23.2 and 24.3±0.2 degrees 2 theta.

In one embodiment, the present invention provides a crystalline daclatasvir dihydrochloride hydrate further characterized by an X-ray powder diffraction (XRPD) spectrum having peak reflections at about 8.3 and 30.6±0.2 degrees 2 theta.

In one embodiment, the present invention provides a crystalline daclatasvir dihydrochloride hydrate characterized by an X-ray powder diffraction (XRPD) spectrum having peak reflections at about 8.3, 9.5, 11.0, 23.2, 24.3 and 30.6±0.2 degrees 2 theta.

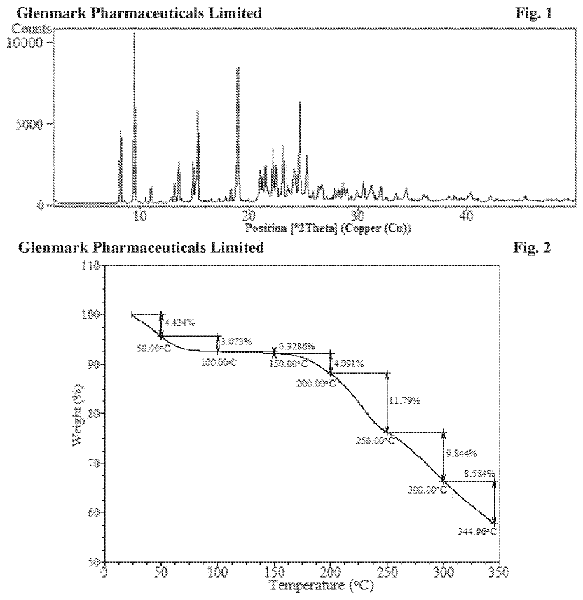

In one embodiment, the present invention provides a crystalline daclatasvir dihydrochloride hydrate characterized by an X-ray powder diffraction (XRPD) spectrum having peak reflections at about 9.5, 11,0, 23.2 and 24.3±0.2 degrees 2 theta which is substantially in accordance with FIG. 1.

In one embodiment, the present invention provides a crystalline daclatasvir dihydrochloride hydrate characterized by TGA thermogram, showing a weight loss of about 1 weight % to 13 weight % up to 100° C. determined over the temperature range of 0° C. to 350° C. and heating rate 10° C./min.

In one embodiment, the present invention provides a crystalline daclatasvir dihydrochloride hydrate characterized by TGA thermogram, showing a weight loss of about 6.5 weight % to 8 weight % up to 100° C. determined over the temperature range of 0° C. to 350° C. and heating rate 10° C./min. which is substantially in accordance with FIG. 2.

In one embodiment, the present invention provides a crystalline daclatasvir dihydrochloride hydrate characterized by DSC thermogram having endothermic peak at about 226±4° C.

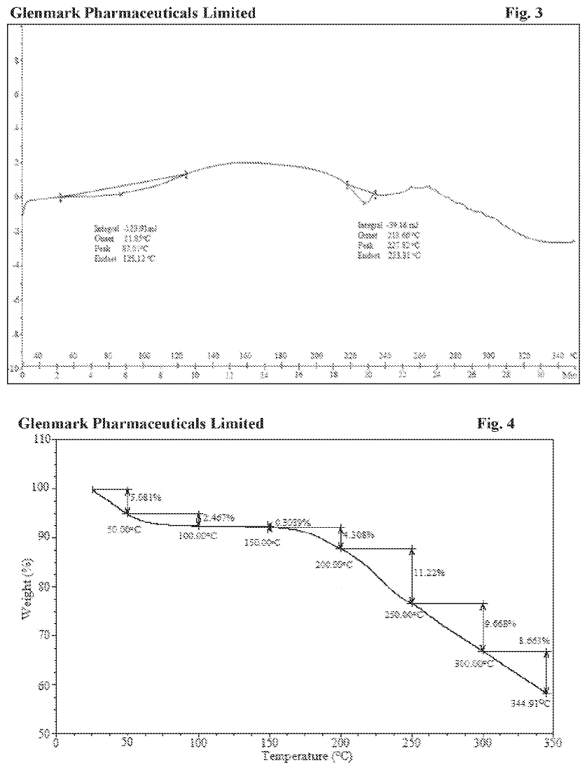

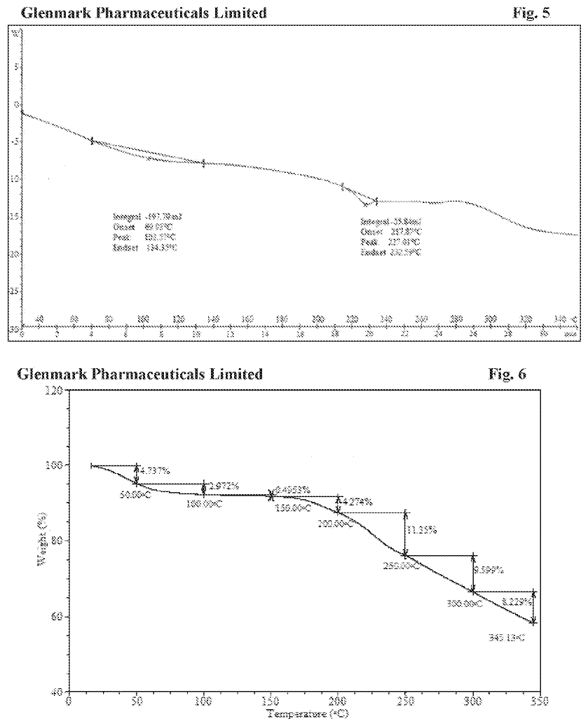

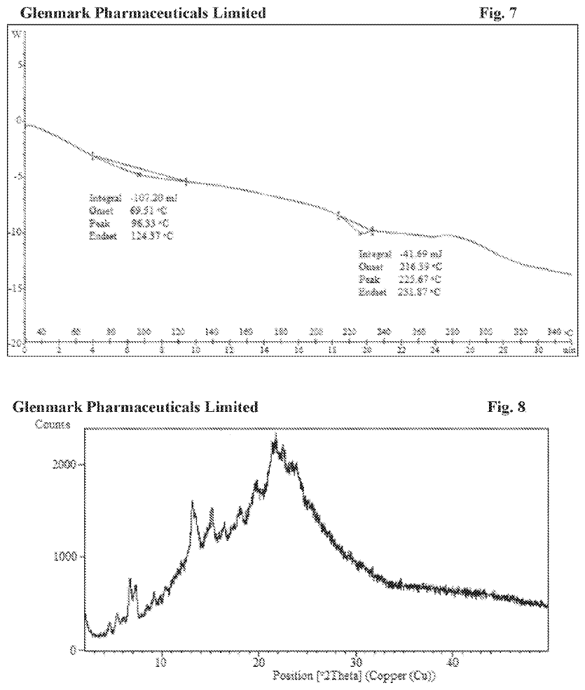

in one embodiment, the present invention provides a crystalline daclatasvir dihydrochloride hydrate characterized by DSC thermogram having endothermic peak at about 227° C. which is substantially in accordance with FIG. 3.

In one embodiment, the present invention provides a crystalline daclatasvir dihydrochloride hydrate characterized by data selected from the group consisting of: an X-ray powder diffraction (XRPD) pattern as depicted in FIG. 1, a TGA thermogram as depicted in FIG. 2; a DSC thermogram as depicted in FIG. 3; and any combination thereof.

In one embodiment, the present invention provides a crystalline daclatasvir dihydrochloride hydrate wherein the water content is in the range of 1-12% by Karl Fischer method.

In one embodiment, the present invention provides a crystalline daclatasvir dihydrochloride hydrate wherein the water content is in the range of 3-10% by Karl Fischer method.

In one embodiment, the present invention provides a crystalline daclatasvir dihydrochloride hydrate wherein the water content is in the range of 7-9% by Karl Fischer method.

In one embodiment, the present invention provides a crystalline daclatasvir dihydrochloride hydrate wherein the water content is in the range of 8-8.5% by Karl Fischer method.

In one embodiment, the present invention provides a crystalline daclatasvir dihydrochloride trihydrate.

In one embodiment, the present invention provides a process for the preparation of crystalline daclatasvir dihydrochloride hydrate, the process comprising:
(a) providing daclatasvir in a solvent or a mixture of solvents;
(b) adding hydrochloric acid, optionally in the presence of a solvent, to the mixture Obtained in step (a) to form daclatasvir dihydrochloride;
(c) optionally isolating daclatasvir dihydrochloride from the mixture of step (b);
(d) providing the daclatasvir dihydrochloride obtained in step (b) or step (c) in water to form a mixture;
(e) obtaining crystalline daclatasvir dihydrochloride hydrate from the mixture of step (d); and
(f) isolating the crystalline daclatasvir dihydrochloride hydrate.

In (a) of the process for the preparation of crystalline daclatasvir dihydrochloride hydrate, daclatasvir is mixed with a solvent or a mixture of solvents.

The solvent includes but is not limited to haloalkanes such as dichloromethane, chloroform, ethylene dichloride, and the like; ketones such as acetone, ethyl methyl ketone, methyl isobutyl ketone and the like; alcohols such as methanol, ethanol, 1-propanol, 2-propanol, 1-butanol, 2-butanol, 1-pentanol, 1-octanol and the like; ethers such as diethyl ether, diisopropyl ether, methyl tert-butyl ether, tetrahydrofuran, dioxane and the like; esters such as methyl acetate, ethyl acetate, n-propyl acetate, isopropyl acetate, butyl acetate, tert-butyl acetate and the like; hydrocarbons such as toluene, xylene, chlorobenzene, heptane, hexane, cyclohexane and the like; dimethyl sulfoxide; dimethyl formamide; dimethyl acetamide; water; or mixtures thereof.

In (b) of the process for the preparation of crystalline daclatasvir dihydrochloride hydrate, hydrochloric acid is added, optionally in the presence of a solvent, to the mixture obtained in step (a) to form daclatasvir dihydrochloride.

The solvent is selected from the group consisting of haloalkanes, ketones, alcohols, ethers, esters, hydrocarbons, water, or mixtures thereof as discussed supra.

The reaction may be carried out at a temperature in the range from about 20° C. to about 100° C. Preferably, the reaction is carried out at about 55° C. to about 90° C. The stirring time may range from about 2 hours to about 7 hours, or longer. The solution may be optionally treated with charcoal and filtered to get a particle-free solution.

In (c) of the process for the preparation of crystalline daclatasvir dihydrochloride hydrate, daclatasvir dihydrochloride is isolated from the mixture of step (b) by any method known in the art. The method, may involve any of techniques, known in the art, including filtration by gravity or by suction, centrifugation, and the like, evaporation by lyophilisation, freeze-drying technique, spray drying, fluid bed drying, flash drying, spin flash drying, thin-film drying, agitated nutsche filter dryer, complete evaporation in, for example, a rotavapor, a vacuum paddle dryer or in a conventional reactor under vacuum, or concentrating the solution, cooling the solution if required and filtering the obtained solid by gravity or by suction, centrifugation, and the like.

In one embodiment, the isolated daclatasvir dihydrochloride is in crystalline form or is in amorphous form.

In (d) of the process for the preparation of crystalline daclatasvir dihydrochloride hydrate, the daclatasvir dihydrochloride obtained in step (b) or step (c) is taken in water to form a mixture.

The reaction may be carried out at a temperature in the range from about 20° C. to about 100° C. Preferably, the reaction is carried out at about 55° C. to about 90° C. The stirring time may range from about 2 hours to about 7 hours, or longer. The solution may be optionally treated with charcoal and filtered to get a particle-free solution.

In (e) of the process for the preparation of crystalline daclatasvir dihydrochloride hydrate, crystalline daclatasvir dihydrochloride hydrate is obtained from the mixture of step (d), the process comprising:
(i) optionally cooling and stirring the solution obtained in step (d); or
(ii) removing the solvent from the solution obtained in step (d); or
(iii) treating the solution of step (d) with an anti-solvent to form a mixture and optionally, cooling and stirring the obtained mixture.
(iv) heating the mixture of step (d) to a temperature of 40° C. to 110° C. optionally maintaining the solution at the temperature of 40° C. to 110° C. and cooling the solution of step (b) or step (c) to a temperature of −5° C. to 35° C.

In (i) of the above process, crystalline daclatasvir dihydrochloride hydrate is Obtained by optionally cooling and stirring the solution obtained in step (d). The stirring time may range from about 2 hours to about 12 hours, or longer. The temperature may range from about 20° C. to about 100° C.

In (ii) of the above process, crystalline daclatasvir dihydrochloride hydrate is obtained by removing the solvent from the solution obtained in step (d). Removal of solvent may be accomplished by substantially complete evaporation of the solvent or concentrating the solution, cooling the solution if required and filtering the obtained solid. The solution may be completely evaporated in, for example, a rotavapor, a vacuum paddle dryer or in a conventional reactor under vacuum above about 720 mm Hg, or evaporated by lyophilisation, freeze-drying technique, spray drying, fluid bed drying, flash drying, spin flash drying, thin-film drying. The solution may also be completely evaporated as discussed supra, adding a second solvent, optionally cooling and stirring the obtained mixture and filtering the obtained solid.

The second solvent includes but is not limited to haloalkanes such as dichloromethane, chloroform, ethylene dichloride, and the like; ketones such as acetone, ethyl methyl ketone, methyl isobutyl ketone and the like; alcohols such as methanol, ethanol, 1-propanol, 2-propanol, 1-butanol, 2-butanol, 1-pentanol, 1-octanol and the like; ethers such as diethyl ether, diisopropyl ether, methyl tert-butyl ether, tetrahydrofuran, dioxane and the like; esters such as methyl acetate, ethyl acetate, n-propyl acetate, isopropyl acetate, butyl acetate, tert-butyl acetate and the like; hydrocarbons such as toluene, xylene, chlorobenzene, heptane, hexane, cyclohexane and the like; dimethyl sulfoxide; dimethyl formamide; dimethyl acetamide; water; or mixtures thereof.

In (iii) of the above process, crystalline daclatasvir dihydrochloride hydrate is obtained by adding an anti-solvent to the solution obtained in step (d) to form a mixture and optionally, cooling and stirring the obtained mixture. The stirring time may range from about 2 hours to about 12 hours, or longer. The temperature may range from about 20° C. to about 100° C.

The anti-solvent is selected such that crystalline daclatasvir dihydrochloride hydrate is precipitated out from the solution.

The anti-solvent includes but is not limited to haloalkanes such as dichloromethane, chloroform, ethylene dichloride, and the like; ketones such as acetone, ethyl methyl ketone, methyl isobutyl ketone and the like; alcohols such as methanol, ethanol, 1-propanol, 2-propanol, 1-butanol, 2-butanol, 1-pentanol, 1-octanol and the like; ethers such as diethyl ether, diisopropyl ether, methyl tert-butyl ether, tetrahydrofuran, dioxane and the like; esters such as methyl acetate, ethyl acetate, n-propyl acetate, isopropyl acetate, butyl acetate, tert-butyl acetate and the like; hydrocarbons such as toluene, xylene, chlorobenzene, heptane, hexane, cyclohexane and the like; dimethyl sulfoxide; dimethyl formamide; dimethyl acetamide; water; or mixtures thereof.

In (iv) of the above process, crystalline daclatasvir dihydrochloride hydrate is obtained by heating the mixture of step (d) to a temperature of about 40° C. to 110° C. and maintaining the temperature for about 2 hours to about 12 hours with or without stirring, followed by cooling with or without stirring at a temperature of −5° C. to 35° C.

The stiffing time may range from about 2 hours to about 12 hours, or longer. The solution may be optionally treated with charcoal and filtered to get a particle-free solution.

In (f) of the process for the preparation of crystalline daclatasvir dihydrochloride hydrate, the crystalline daclatasvir dihydrochloride hydrate is isolated by any method known in the art. The method, may involve any of techniques, known in the art, including filtration by gravity or by suction, centrifugation, and the like, complete evaporation in, for example, a rotavapor, a vacuum paddle dryer or in a conventional reactor under vacuum, or concentrating the solution, cooling the solution if required and filtering the obtained solid by gravity or by suction, centrifugation, and the like.

In one embodiment, the present invention provides a process for the preparation of crystalline daclatasvir dihydrochloride hydrate, the process comprising:
(a) providing daclatasvir in water, optionally in the presence of a solvent;
(b) adding hydrochloric acid, optionally in the presence of a solvent, to the mixture obtained in step (a) to form daclatasvir dihydrochloride;
(c) obtaining crystalline daclatasvir dihydrochloride hydrate from the mixture of step (b); and
(d) isolating the crystalline daclatasvir dihydrochloride hydrate.

In step (b) of the above process, daclatasvir dihydrochloride is not isolated from the mixture of step (b).

In one embodiment, the present invention provides a process for the preparation of crystalline daclatasvir dihydrochloride hydrate, the process comprising:
(a) reacting daclatasvir with hydrochloric acid, to obtain daclatasvir hydrochloride;
(b) dissolving daclatasvir di hydrochloride in water to obtain a solution;
(c) heating the solution containing to a temperature in the range of 40-70° C.;
(d) stirring the solution at a temperature in the range of 40-70° C. for a period of 1-12 hours;
(e) cooling the reaction mass of step d; and
(f) isolating the crystalline daclatasvir dihydrochloride hydrate.

In one embodiment, the present invention provides a process for the preparation of crystalline daclatasvir dihydrochloride hydrate, the process comprising:
(a) dissolving daclatasvir dihydrochloride in water, optionally in the presence of a solvent, to form a solution;
(b) heating the solution obtained in step (a) to a temperature of about 40° C. to 110° C.;
(c) optionally maintaining the solution of step (b) at the temperature of about 40° C. to 110° C.;
(d) cooling the solution of step (b) or step (c) to a temperature of about −5° C. to 35° C.; and
(e) isolating the crystalline daclatasvir dihydrochloride hydrate.

In (a) of the process for the preparation of crystalline daclatasvir dihydrochloride hydrate, daclatasvir dihydrochloride is dissolved in water, optionally in the presence of a solvent, to form a solution.

The solvent includes but is not limited to haloalkanes such as dichloromethane, chloroform, ethylene dichloride, and the like; ketones such as acetone, ethyl methyl ketone, methyl isobutyl ketone and the like; alcohols such as methanol, ethanol, 1-propanol, 2-propanol, 1-butanol, 2-butanol, 1-pentanol, 1-octanol and the like; ethers such as diethyl ether, diisopropyl ether, methyl tert-butyl ether, tetrahydrofuran, dioxane and the like; esters such as methyl acetate, ethyl acetate, n-propyl acetate, isopropyl acetate, butyl acetate, tert-butyl acetate and the like; hydrocarbons such as toluene, xylene, chlorobenzene, heptane, hexane, cyclohexane and the like; dimethyl sulfoxide; dimethyl formamide; dimethyl acetamide; water; or mixtures thereof.

The solution may be optionally treated with charcoal and filtered to get a particle-free solution.

In (b) of the process for the preparation of crystalline daclatasvir dihydrochloride hydrate, the solution obtained in step (a) is heated to a temperature of about 40° C. to 110° C.

The stirring time may range from about 2 hours to about 12 hours, or longer. The solution may be optionally treated with charcoal and filtered to get a particle-free solution.

In (c) of the process for the preparation of crystalline daclatasvir dihydrochloride hydrate, the solution of step (b) is maintained at the temperature of 40° C. to 110° C.

The time may range from about 2 hours to about 12 hours, or longer. The solution may be optionally treated with charcoal and filtered to get a particle-free solution.

In (d) of the process for the preparation of crystalline daclatasvir dihydrochloride hydrate, the solution of step (b) or step (c) is cooled to a temperature of −5° C. to 35° C.

The stirring time may range from about 2 hours to about 12 hours, or longer. The solution may be optionally treated with charcoal and filtered to get a particle-free solution.

In (e) of the process for the preparation of crystalline daclatasvir dihydrochloride hydrate, the crystalline daclatasvir dihydrochloride hydrate is isolated by any method known in the art. The method, may involve any of techniques, known in the art, including filtration by gravity or by suction, centrifugation, and the like, complete evaporation in, for example, a rotavapor, a vacuum paddle dryer or in a conventional reactor under vacuum, or concentrating the solution, cooling the solution if required and filtering the obtained solid by gravity or by suction, centrifugation, and the like.

The isolated crystalline daclatasvir dihydrochloride hydrate may be dried in an oven, air tray dryer, vacuum tray dryer, rotary dryer, rotary vacuum dryer, flash dryer, spin flash dryer, fluid bed dryer, and the like.

The drying may be carried out for a period of 2 hours to 60 hours.

In one embodiment, the isolated crystalline daclatasvir dihydrochloride hydrate may be dried by a process comprising:
(a) drying the isolated crystalline daclatasvir dihydrochloride hydrate of step (e) at about 25° C. to about 60° C. in vacuum tray dryer for about 12 hours to about 16 hours; or
(b) drying the isolated crystalline daclatasvir dihydrochloride hydrate of step (e) at about 30° C. to about 35° C. in air oven for about 12 hours to about 16 hours; or
(c) drying the isolated crystalline daclatasvir dihydrochloride hydrate of step (e) at about room temperature on rotavapor under vacuum for about 12 hours to about 16 hours.

In one embodiment, the present invention provides a process for the preparation of crystalline daclatasvir dihydrochloride hydrate by aqueous recrystallization of daclatasvir dihydrochloride.

In one embodiment, the present invention provides a process for the preparation of crystalline daclatasvir dihydrochloride hydrate, the process comprising:
(a) dissolving daclatasvir dihydrochloride in water, optionally in the presence of a solvent, to form a solution; and
(b) recovering the crystalline daclatasvir dihydrochloride hydrate from the solution of step (a).

In step (a) of the above process, the reaction temperature may be in the range of about 20° C. to about 110° C.

In step (b) of the above process, the reaction temperature may be in the range of about −5° C. to about 35° C.

In one embodiment, the present invention provides a process for the preparation of crystalline daclatasvir dihydrochloride hydrate, the process comprising:
(a) providing daclatasvir in water and organic solvent to form a mixture;
(b) adding hydrochloric acid, to the mixture obtained in step (a) to form daclatasvir dihydrochloride;
(c) separating the organic layer from the aqueous layer;
(d) partially distilling the aqueous layer and stirring the aqueous layer at a temperature in the range of 40-60° C.;
(e) cooling the reaction mass at a temperature in the range of 10-30° C.; and
(f) isolating the crystalline daclatasvir dihydrochloride hydrate.

In one embodiment, the present invention provides a process for the preparation of crystalline daclatasvir dihydrochloride hydrate, the process comprising:
(a) providing daclatasvir in water and optionally an organic solvent to form a mixture;
(b) adding hydrochloric acid, to the mixture obtained in step (a) to form daclatasvir dihydrochloride;
(c) separating the organic layer from the aqueous layer;
(d) stirring the aqueous layer at a temperature in the range of 40-60° C.;
(e) optionally cooling the reaction mass at a temperature in the range of 10-30° C.; and
(f) isolating the crystalline daclatasvir dihydrochloride hydrate.

The present invention provides crystalline daclatasvir dihydrochloride hydrate, obtained by the above processes, as characterized and analyzed by following techniques:
A. X-ray powder diffraction profiles were obtained using an X-ray Diffractometer (Philips X′Pert Pro, PANalytical). The measurements were carried out with a Pre FIX module programmable divergence slit and anti-scatter Slit (Offset 0.00°) ; target, Cu; filter, Ni; detector, X′Celerator; Scanning Mode; Active length (2Theta)=2.122°; generator 45KV; tube current 40 mAmp. The samples were scanned in the full 2θ range of 2-50° with a “time-per-step” optimized to 50 sec.
B. DSC (Mettler Toledo 822e): Temperature range is “30° C. to 350° C.” and heating rate is 10° C./minute.
C. Thermo Gravimetric Analyzer: TGA Q500 V6.5. Thermogram was recorded at 30-350° C. at the rate of 10° C./min.
D. Karl Fischer: The water content was calculated by the following formula:

$$Water content ( \% ) = \frac{Burette reading \times KF Factor}{Weight of sample in mg} \times 100$$

In one embodiment the present invention provides daclatasvir dihydrochloride hydrate, which has enhanced stability to heat and humidity. According to the present invention, the obtained daclatasvir dihydrochloride hydrate is subjected to accelerated stability at 40° C. and relative humidity (RH) 75%. The daclatasvir dihydrochloride hydrate is found to be chemically and physically very stable. There is no substantial increase in the moisture content that is observed. The XRPD pattern remains the same as initial and no degradation is observed in HPLC.

### Solid State Stability

The solid state stability of the daclatasvir dihydrochloride hydrate was determined by storing approximately 3.0 g of the sample at accelerated stress conditions (40° C./75% relative humidity) for 15 days, 1 month, 3 months, 6 months. The samples were tested by XRPD and HPLC for final purity and degradation products. The results are given in the following Table 1.

TABLE 1

| Conditions | XRPD | HPLC Purity (%) |
|---|---|---|
| After 15 days | Same as initial | 99.9 |
| After 1 month | Same as initial | 99.9 |
| After 3 months | Same as initial | 99.89 |
| After 6 months | Same as initial | 99.88 |

The daclatasvir dihydrochloride hydrate of the present invention was found to be chemically and physically very stable. There is no substantial increase in the moisture content that was observed. The XRPD pattern remains same as initial and there is no degradation observed in HPLC.

### Physical Stability

The physical stability of the daclatasvir dihydrochloride hydrate, of the present invention was determined by storing approximately 3.0 g of the sample a) at 50° C. b) at 70° C. c) at 100° C. The samples were tested by XRPD and Karl-Fisher titrator after 15 hours and 72 hours. There was no change in form observed in daclatasvir dihydrochloride hydrate of the present invention under both drying and humid conditions. The results are shown in the following Table 2.

TABLE 2

| | Results | |
|---|---|---|
| Conditions | Key XRPD peaks | KF |
| Initial form | 8.3, 9.5, 11.1, 23.2, 24.3, 30.6 | 7.99 |
| Drying at 50° C. for 15 hours | There was no change in XRPD | 8.03 |
| Drying at 50° C. for 72 hours | pattern and no additional peaks | 8.12 |
| Drying at 70° C. for 15 hours | were observed. | 8.03 |
| Drying at 70° C. for 72 hours | | 8.02 |
| Drying at 100° C. for 15 hours | | 8.00 |

### Transformation Kinetics

The study was carried out by slurrying 1 g of daclatasvir dihydrochloride hydrate of the present invention and literature Form N-2 of daclatasvir dihydrochloride, in water at 25 to 30° C. for 24 hours. The solid samples were then characterized by XRPD. No discernible form conversion or dissociation was observed in water for daclatasvir dihydrochloride hydrate of the present invention, but literature form N-2, did not retain its form and was converted to the daclatasvir dihydrochloride hydrate of the present invention. This clearly shows that literature form N-2 lacks aqueous stability and is not able to maintain its form, whereas the form of the present invention is stable.

TABLE 3
Conditions- Slurrying in water at 25 to 30° C. for 24 hours

| Initial Sample | Result |
|---|---|
| Form-N2 | Converted to crystalline daclatasvir |
| | dihydrochloride hydrate |
| Crystalline daclatasvir | There was no change in XRPD pattern and |
| hydrochloride hydrate | no additional peaks were observed. |

Hygroscopicity

The study was carried out by subjecting 4 g of daclatasvir dihydrochloride hydrate of the present invention and literature Form N-2 of daclatasvir dihydrochloride to 25′C/60% relative humidity and 40′C/75% relative humidity. About 100 mg of sample was withdrawn each time at timely interval of 4, 12, 24 and 48 hours from the conditions is and tested for description and water content by Karl Fischer. The results are shown in the following Table 4 and 5.

TABLE 4
Daclatasvir dihydrochloride hydrate of the present invention

| Time point | | | | |
|---|---|---|---|---|
| | 4 | 12 | 24 | 48 |
| Initial | hours | hours | hours | hours |

25° C./60% relative humidity

| | | | | | |
|---|---|---|---|---|---|
| Description | Creamish | Creamish | Creamish | Creamish | Creamish |
| | powder | powder | powder | powder | powder |
| Moisture | 8.76% | 8.77% | 8.86% | 8.89% | 8.91% |
| content | | | | | |
| by KF | | | | | |

40° C./75% relative humidity

| | | | | | |
|---|---|---|---|---|---|
| Description | Creamish | Creamish | Creamish | Creamish | Creamish |
| | powder | powder | powder | powder | powder |
| Moisture | 8.76% | 8.76% | 8.81% | 8.86% | 8.92% |
| content | | | | | |
| by KF | | | | | |

TABLE 5
Daclatasvir dihydrochloride literature Form N-2

| Time point | | | | |
|---|---|---|---|---|
| | 4 | 12 | 24 | 48 |
| Initial | hours | hours | hours | hours |

25° C/60% relative humidity

| | | | | | |
|---|---|---|---|---|---|
| Description | Creamish | Creamish | Creamish | Creamish | Creamish |
| | powder | powder | powder | powder | powder |
| Moisture | 2.12% | 2.45% | 2.47% | 2.44% | 2.47% |
| content | | | | | |
| by KF | | | | | |

40° C./75% relative humidity

| | | | | | |
|---|---|---|---|---|---|
| Description | Creamish | Creamish | Creamish | Creamish | Creamish |
| | powder | powder | powder | powder | powder |
| Moisture | 2.12% | 2.52% | 2.54% | 2.65% | 2.77% |
| content | | | | | |
| by KF | | | | | |

This study suggests that daclatasvir dihydrochloride hydrate of the present invention is non-hygroscopic whereas the literature form N-2 is more hygroscopic than the form of the present invention.

### Photostability

This study was carried out in a photostability testing chamber by subjecting daclatasvir dihydrochloride hydrate of the present invention to irradiation with light of 4000 lux at 25° C. for 2 weeks and the samples were tested for XRPD and stability. The XRPD pattern remains same as initial and there is no degradation Observed in HPLC.

### Scanning Electron Microscopy (SEM)

SEM was used to obtain more information about the surface and the morphology. The daclatasvir dihydrochloride hydrate of the present invention was brick shape, whereas literature from N-2 was granular in shape.

### Dynamic Vapor System

Sorption/desorption isotherms were measured using the Dynamic vapor System. Measurement was carried out at 25° C. and Relative humidity of 40% to 90%.

Adsorption desorption profile for the crystalline daclatasvir dihydrochloride hydrate form of the present invention is reversible which indicates that the crystal form of the present invention is stable. Stable crystalline materials exhibit this type of profile.

In one embodiment, the present invention provides a salt of daclatsavir with malonic acid.

In one embodiment, the present invention provides the salt of daclatasvir with malonic acid, characterized by a proton NMR spectrum having peak positions at 11.79 (br, 2H), 7.78-7.80 (dd, 4H), 7.69-7.71 (dd, 4H), 7.55 (s, 2H), 7.31-7.33 (d, 2H), 5.08-5.10 (t, 2H), 4.08 (t, 2H), 3.8 (m, 4H) 3.54 (s, 6H), 3.2 (s, 4H), 1.95-2.17 (m, 10), 0.79-0.91 (d, 6H), 0.84-0.86 (d, 6H).

In one embodiment, the present invention provides crystalline daclatasvir malonate characterized by an X-ray powder diffraction (XRPD) spectrum having peak reflections at about 6.7, 7.3, 13.1, 15.1 21.6±0.2 degrees 2 theta.

In one embodiment, the present invention provides crystalline daclatasvir malonate characterized by an X-ray powder diffraction (XRPD) spectrum having peak reflections as listed in below table.

| Pos. [°2Th.] ± 0.2° θ | d-spacing [Å] | Rel. Int. [%] |
|---|---|---|
| 4.5 | 19.51 | 15.72 |
| 5.3 | 16.54 | 22.32 |
| 6.7 | 13.14 | 63.06 |
| 7.3 | 12.06 | 47.08 |
| 9.1 | 9.65 | 17.59 |
| 10.3 | 8.53 | 8.87 |
| 13.1 | 6.75 | 91.75 |
| 15.1 | 5.84 | 60.28 |
| 16.4 | 5.39 | 24.05 |
| 18.1 | 4.88 | 28.49 |
| 19.6 | 4.50 | 47.43 |
| 21.6 | 4.11 | 100.00 |
| 22.5 | 3.94 | 95.47 |
| 23.86 | 3.72 | 74.91 |

In one embodiment, the present invention provides a process for preparation of daclatasvir dihydrochloride comprising
a. reacting daclatasvir with acid to form daclatasvir acid addition salt;
b. reacting daclatasvir acid addition salt with a base to form daclatasvir;
c. reacting the daclatasvir of step b with hydrochloric acid to form daclatasvir hydrochloride.

The acid may be selected from inorganic or organic acids like mandelic acid, succinic acid, oxalic acid, malonic acid.

In one embodiment, the present invention provides a process for preparation of daclatasvir dihydrochloride comprising
a. reacting daclatasvir with malonic acid to form daclatasvir malonate;
b. reacting daclatasvir malonate with a base to form daclatasvir;
c. reacting the daclatasvir of step b with hydrochloric acid to form daclatasvir hydrochloride.

In one embodiment, the present invention provides use of daclatasvir malonate to prepare daclatasvir dihydrochloride.

In one embodiment, the present invention provides a process for daclatasvir dihydrochloride comprising
a. preparing daclatasvir malonate; and
b. converting daclatasvir malonate to daclatasvir hydrochloride.

In one embodiment, the present invention provides a process for preparation of crystalline daclatasvir dihydrochloride hydrate comprising
a. reacting daclatasvir with malonic acid to form daclatasvir malonate;
b. reacting daclatasvir malonate with a base to form daclatasvir;
c. reacting the daclatasvir of step b with hydrochloric acid to form daclatasvir hydrochloride;
d. dissolving daclatasvir dihydrochloride in water, optionally in the presence of a solvent, to form a solution;
e. heating the solution obtained in step (d) to a temperature of about 40° C. to 110° C.;
f. optionally maintaining the solution of step (e) at the temperature of about 40° C. to 110° C.;
g. cooling the solution of step (e) or step (f) to a temperature of about −5° C. to 35° C.; and
h. isolating the crystalline daclatasvir dihydrochloride hydrate.

In one embodiment, the present invention provides daclatasvir or daclatasvir dihydrochloride or daclatasvir dihydrochloride hydrate, wherein level of below impurities is less than 0.15% w/w as determined by HPLC.

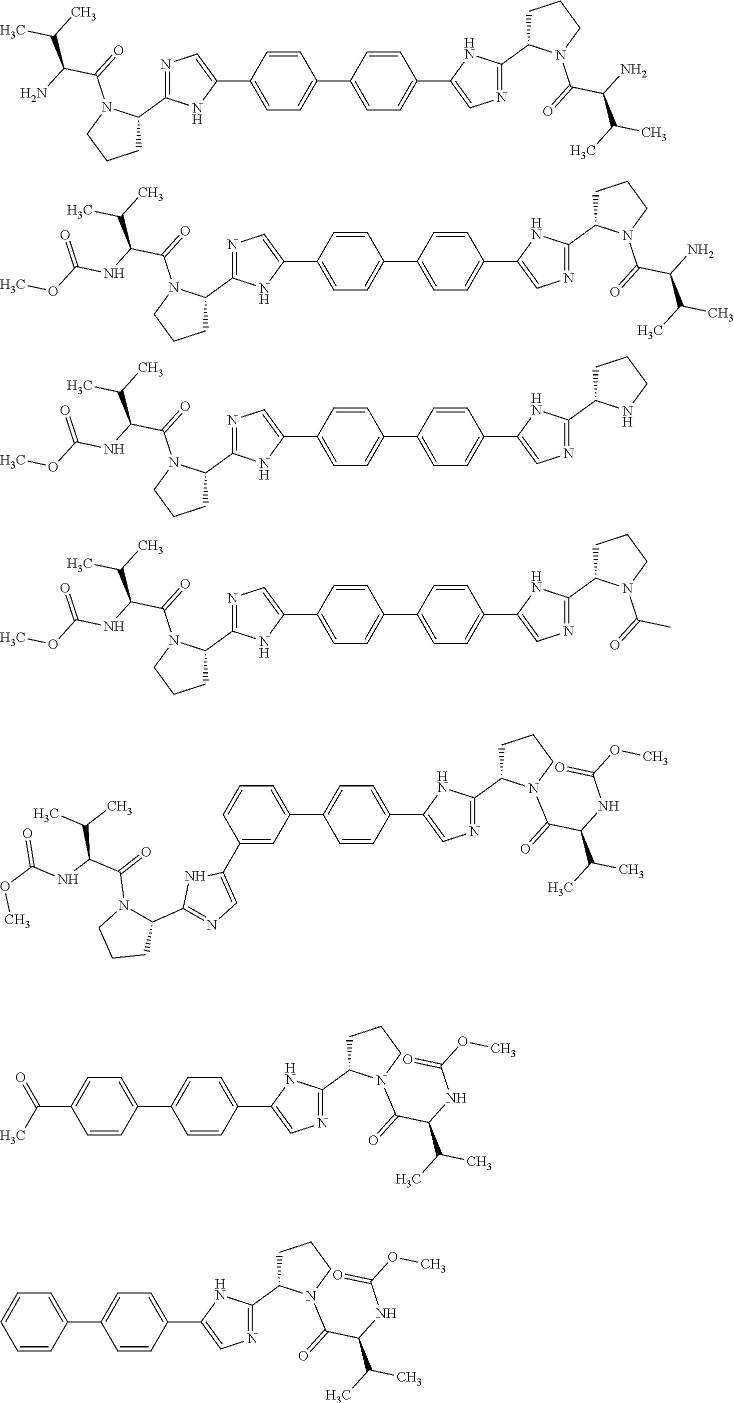

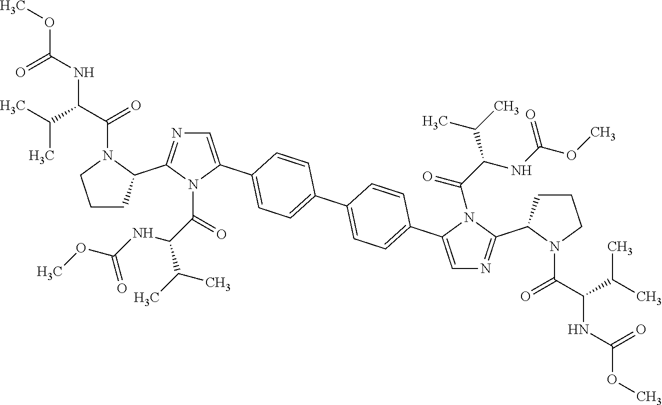 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)n4C(=O)[C@@H](NC(=O)OC)C(C)C)cc3)cc2)n1C(=O)[C@@H](NC(=O)OC)C(C)C)C(C)C]

### Other Stereoisomers of Daclatasvir

1. methyl N-[(2S)-1-[(2R)-2-{5-[4-(4-{2-[(2R)-1-[(2S)-2-[(methoxycarbonyl)amino]-3-methylbutanoyl]pyrrolidin-2-yl]-1H-imidazol-5-yl}phenyl)phenyl]-1H-imidazol-2-yl}pyrrolidin-1-yl]-3-methyl-1-oxobutan-2-yl]carbamate (SRRS isomer).
2. methyl N-[(2S)-1-[(2R)-2-{5-[4-(4-{2-[(2S)-1-[(2R)-2-[(methoxycarbonyl)amino]-3-methylbutanoyl]pyrrolidin-2-yl]-1H-imidazol-5-yl}phenyl)phenyl]-1H-imidazol-2-yl}pyrrolidin-1-yl]-3-methyl-1-oxobutan-2-yl]carbamate (RSSR Isomer).
3. methyl N-[(2R)-1-[(2S)-2-{5-[4-(4-{2-[(2S)-1-[(2R)-2-[(methoxycarbonyl)amino]-3-methylbutanoyl]pyrrolidin-2-yl]-1H-imidazol-5-yl}phenyl)phenyl]-1H-imidazol-2-yl}pyrrolidin-1-yl]-3-methyl-1-oxobutan-2-yl]carbamate (RSSR Isomer).
4. methyl N-[(2S)-1-[(2R)-2-{5-[4-(4-{2-[(2R)-1-[(2R)-2-[(methoxycarbonyl)amino]-3-methylbutanoyl]pyrrolidin-2-yl]-1H-imidazol-5-yl}phenyl)phenyl]-1H-imidazol-2-yl}pyrrolidin-1-yl]-3-methyl-1-oxobutan-2-yl]carbamate (RRRS Isomer).
5. methyl N-[(2R)-1-[(2R)-2-{5-[4-(4-{2-[(2S)-1-[(2R)-2-[(methoxycarbonyl)amino]-3-methylbutanoyl]pyrrolidin-2-yl]-1H-imidazol-5-yl}phenyl)phenyl]-1H-imidazol-2-yl}pyrrolidin-1-yl]-3-methyl-1-oxobutan-2-yl]carbamate (RSRR Isomer).
6. methyl N-[(2S)-1-[(2S)-2-{5-[4-(4-{2-[(2S)-1-[(2R)-2-[(methoxycarbonyl)amino]-3-methylbutanoyl]pyrrolidin-2-yl]-1H-imidazol-5-yl}phenyl)phenyl]-1H-imidazol-2-yl}pyrrolidin-1-yl]-3-methyl-1-oxobutan-2-yl]carbamate (RSSS Isomer).
7. methyl N-[(2S)-1-[(2R)-2-{5-[4-(4-{2-[(2S)-1-[(2S)-2-[(methoxycarbonyl)amino]-3-methylbutanoyl]pyrrolidin-2-yl]-1H-imidazol-5-yl}phenyl)phenyl]-1H-imidazol-2-yl}pyrrolidin-1-yl]-3-methyl-1-oxobutan-2-yl]carbamate (SSRS Isomer).

In one embodiment, the present invention provides daclatasvir or daclatasvir dihydrochloride or daclatasvir dihydrochloride hydrate, wherein level of steroisomeric impurities is less than 0.05%.

In one embodiment, the present invention provides mixed solvates of daclatasvir.

The mixed solvates of daclatasvir includes mixed solvates with water, methanol, ethanol, n-propanol, isopropanol, n-butanol, isobutanol, tert-butanol, ethylene glycol, ethyl acetate, n-butyl acetate, isobutyl acetate, acetonitrile, acetone, butanone, methyl isobutyl ketone, tetrahydrofuran, 2-methyl tetrahydrofuran, dioxane, chloroform, dichloromethane, hexane, n-heptane, toluene, N-methyl pyrrolidone, dimethyl formamide or dimethyl sulfoxide.

The present invention provides a process for the preparation of mixed solvate of daclatasvir, the process comprising:
a. dissolving daclatasvir in a suitable solvent, optionally in presence of additional solvent, to form a solution;
b. obtaining mixed solvate of daclatasvir from the solution of step (a); and
c. isolating the mixed solvate of daclatasvir.

The suitable solvent includes water, n-propanol, isopropanol, n-butanol, isobutanol, tert-butanol, ethylene glycol, ethyl acetate, n-butyl acetate, isobutyl acetate, methyl isobutyl ketone, dioxane, chloroform, dichloromethane, hexane, n-heptane, toluene, N-methyl pyrrolidone, dimethyl formamide or dimethyl sulfoxide.

The additional solvent includes but is not limited to esters such as methyl acetate, ethyl acetate, n-propyl acetate, isopropyl acetate, tert-butyl acetate and the like; hydrocarbons such as toluene, xylene, chlorobenzene, heptane, hexane and the like; ethers such as diethyl ether, diisopropyl ether, methyl tert-butyl ether, tetrahydrofuran, dioxane and the like; ketones such as acetone, ethyl methyl ketone, methyl isobutyl ketone and the like; alcohols such as methanol, ethanol, 1-propanol, 2-propanol, 1-butanol, 2-butanol, 1-pentanol, 1-octanol and the like; haloalkanes such as dichloromethane, chloroform, ethylene dichloride, and the like; dimethyl sulfoxide; dimethyl acetamide; water; or mixtures thereof.

In (b) of the above process, the mixed solvate of daclatasvir is obtained from the solution of step (a), the process comprising:
(i) cooling and stirring the solution obtained in (a); or
(ii) removing the solvent from the solution obtained in (a); or
(iii) treating the solution of step (a) with an anti-solvent to form a mixture and optionally, cooling and stifling the obtained mixture.

The anti-solvent is selected such that the mixed solvate of daclatasvir is precipitated out from the solution.

The anti-solvent includes but is not limited to esters such as methyl acetate, ethyl acetate, n-propyl acetate, isopropyl acetate, tea-butyl acetate and the like; hydrocarbons such as toluene, xylene, chlorobenzene, heptane, hexane and the like; ethers such as diethyl ether, diisopropyl ether, methyl tert-butyl ether, tetrahydrofuran, dioxane and the like; ketones such as acetone, ethyl methyl ketone, methyl isobutyl ketone and the like; alcohols such as methanol, ethanol, 1-propanol, 2-propanol, 1-butanol, 2-butanol, 1-pentanol, 1-octanol and the like; haloalkanes such as dichloromethane, chloroform, ethylene dichloride, and the like; dimethyl sulfoxide; dimethyl acetamide; water; or mixtures thereof.

In (c) of the above process, the mixed solvate of daclatasvir is isolated by any method known in the art. The method, may involve any of techniques, known in the art, including filtration by gravity or by suction, centrifugation, and the like, evaporation by lyophilisation, freeze-drying technique, spray drying, fluid bed drying, flash drying, spin flash drying, thin-film drying, agitated nutsche filter dryer, complete evaporation in, for example, a rotavapor, a vacuum paddle dryer or in a conventional reactor under vacuum, or concentrating the solution, cooling the solution if required and filtering the obtained solid by gravity or by suction, centrifugation, and the like.

In one embodiment, the present invention provides mixed solvates of daclatasvir dihydrochloride.

The mixed solvates of daclatasvir dihydrochloride includes mixed solvates with water, methanol, ethanol, n-propanol, isopropanol, n-butanol, isobutanol, tert-butanol, ethylene glycol, ethyl acetate, n-butyl acetate, isobutyl acetate, acetonitrile, acetone, butanone, methyl isobutyl ketone, tetrahydrofuran, 2-methyl tetrahydrofuran, dioxane, chloroform, dichloromethane, hexane, n-heptane, toluene, N-methyl pyrrolidone, dimethyl formamide or dimethyl sulfoxide.

The present invention provides a process for the preparation of mixed solvate of daclatasvir dihydrochloride, the process comprising:
(a) dissolving daclatasvir dihydrochloride in a suitable solvent, optionally in presence of additional solvent, to form a solution;
(b) obtaining mixed solvate of daclatasvir dihydrochloride from the solution of step (a); and
(c) isolating the mixed solvate of daclatasvir dihydrochloride.

The process steps (a) (b) and (c) are as discussed supra for the preparation of mixed solvate of daclatasvir.

In one embodiment, the present invention provides daclatasvir hydrochloride hydrate having a D₅₀ of 16 microns and D₉₀ particle size of less than about 60 microns.

In one embodiment, the present invention provides pharmaceutical compositions comprising daclatasvir or salt, solvate thereof obtained by the processes herein described, having a D₅₀ and D₉₀ particle size of less than about 150 microns, preferably less than about 100 microns, more preferably less than about 50 microns, still more preferably less than about 20 microns, still more preferably less than about 15 microns and most preferably less than about 10 microns. The particle size disclosed here can be obtained by, for example, any milling, grinding, micronizing or other particle size reduction method known in the art to bring the solid state daclatasvir or salt, solvate thereof into any of the foregoing desired particle size range.

The examples that follow are provided to enable one skilled in the art to practice the invention and are merely illustrative of the invention. The examples should not be read as limiting the scope of the invention as defined in the features and advantages.

### EXAMPLES

### Example 1

### Preparation of 1,2-pyrrolidinedicarboxylic acid, 2,2′-[[1,1′-biphenyl]-4,4′-diyibis(2-oxo-2,1-ethanediyl)]bis[1-(1,1-dimethylethyl)]ester, (2S)—

To a stirred mixture of 4,4′-bis(2-bromoacetyl)biphenyl (100 g) in acetonitrile (900 mL) was added N-Boc-L-proline (114 g) and diisopropylethylamine (68.5 g) at about 20° C. to about 30° C. The reaction mass was stirred for about 4 h to about 5 h and quenched with sodium chloride solution. The reaction mixture was stirred and the two layers were separated. The organic layer was washed with sodium chloride solution and concentrated under vacuum till approximately 1 volume remains. Toluene (8 volumes) was added and the solvent was concentrated till 5 volumes remain. The toluene solution is directly used in the next step.
Optional isolation of product:
The organic layer from above example was concentrated under vacuum till approximately 1 volume remains. Ethyl acetate (7 volumes) was added to it and the solvent was concentrated till 5 volumes remain. The organic layer was washed with 5% hydrochloric acid solution and then with water. The organic layer was concentrated under vacuum to give a residue which was degased to give solid.
Yield: 150 g

### Example 2

### Preparation of 1,2-pyrrolidinedicarboxylic acid, 2,2′-[[1,1′-biphenyl]-4,4′-diylbis(2-oxo-2,1-ethanediyl)]bis[1-(1,1-dimethylethyl)]ester, (2S)—

To a stirred mixture of 4,4′-bis(2-bromoacetyl)biphenyl (100 g) in methylene dichloride (900 mL) was added N-Boc-L-proline (114 g) and diisopropylethylamine (68.5 g) at about 20° C. to about 30° C. The reaction mass was stirred for about 4 h to about 5 h and quenched with sodium chloride solution. The reaction mixture was stirred and the two layers were separated. The organic layer was washed with hydrochloric acid solution and water and concentrated under vacuum to give a residue which was degased to give solid.
Yield: 150 g

### Example 3

### Preparation of 1-pyrrolidinecarboxylic acid, 2,2′-([1,1′-biphenyl]-4,4′-diyldi-1H-imidazole-5,2-diyl)bis-1,1′-bis(1,1-dimethylethyl) ester, (2S,2′S)—

Ammonium acetate (390 g) was added to the toluene solution of example 1 and the reaction mixture was stirred, heated to about 95° C. to about 105° C. and maintained for about 10 h to about 15 h. The reaction mixture was cooled to about 75° C. to about 80° C. and water (400 mL) was added to it while maintaining the temperature at about above 75° C. The reaction mixture was stirred and the two layers were separated at about the same temperature. The organic layer was cooled to about room temperature and stirred for about 2 h to about 3 h. The solid obtained was filtered, washed with toluene and dried under vacuum at about 45° C. to about 55° C. Yield: 130 g

### Example 4

### Preparation of 5,5′-(4,4′-biphenyldiyl)bis(2-((2S)-2-pyrrolidinyl)-1H-imidazole)tetrahydrochloride

To a stirred mixture of 1-pyrrolidinecarboxylic acid, 2,2′-([1,1′-biphenyl]-4,4′-diyldi-1H-imidazole-5,2-diyl)bis-, 1,1′-bis(1,1-dimethylethyl)ester, (2S,2′S)— (60 g) in isopropyl alcohol (180 mL) was added water (90 mL) followed by hydrochloric acid (90 mL). The reaction mixture was heated to about 50° C. to about 55° C. and maintained at about the same temperature for about 3 h to 5 h. Isopropyl alcohol (720 mL) was added to the reaction mixture which was cooled to about 20° C. to about 30° C. and stirred for about 2 h to about 3 h. The solid obtained was filtered and washed with isopropyl alcohol. The wet cake was added into methanol and the reaction mixture was heated to about 50° C. to about 55° C. under stirring and maintained for about 1 h at about the same temperature. The reaction mixture was cooled to about 20° C. to about 30° C. and stirred for about 2 h to about 3 h. The solid was filtered, washed with methanol and dried under vacuum at about 45° C. to about 55° C. Yield: 130 g

### Example 5

### Preparation of 5,5′-(4,4′-biphenyldiyl)bis(2-((2S)-2-pyrrolidinyl)-1H-imidazole)tetrahydrochloride

To a stirred mixture of 1-pyrrolidinecarboxylic acid, 2,2′-([1,1′-biphenyl]-4,4′-diyldi-1H-imidazole-5,2-diyl)bis-, 1,1′-bis(1,1-dimethylethyl) ester, (2S,2′S)— (60 g) in ethyl acetate (180 mL) was added hydrochloric acid in ethyl acetate (100 mL). The reaction mixture was heated to about 50° C. to about 55° C. and maintained at about the same temperature for about 3 h to 5 h. The reaction mixture was cooled to about 20° C. to about 30° C. and stirred for about 2 h to about 3 h. The solid obtained was filtered and washed with ethyl acetate. The wet cake was added into methanol and the reaction mixture was heated to about 50° C. to about 55° C. under stirring and maintained for about 1 h at about the same temperature. The reaction mixture was cooled to about 20° C. to about 30° C. and stirred for about 2 h to about 3 h. The solid was filtered, washed with methanol and dried under vacuum at about 45° C. to about 55° C. Yield: 130 g

### Example 6

### Preparation of Daclatasvir Dihydrochloride

To a stirred mixture of Moc-L-valine (7.5 g) in acetonitrile (100 mL) was added 1-hydroxy-7-azabenzotriazole (HOAt) (5.84 g) and 1-ethyl -3-(3-dimethylaminopropyl)carbodiimide hydrochloride (EDC.HCl) (8.2 g) and the reaction mixture was stirred for about 1 h. 5,5′-(4,4′-biphenyldiyl)bis(2-((2S)-2-pyrrolidinyl)-1H-imidazole)tetrahydrochloride (10.2 g) was added. The reaction mixture was cooled to about 0° C. to about 10° C. and diisopropylethylamine (9.2 g) was added to it. The reaction mixture was maintained at about 5° C. to about 15° C. for about 12 h to about 16 h. Water and ethyl acetate were added to the reaction mixture which was stirred for about 15 min. The two layers were separated and the aqueous layer was extracted with ethyl acetate. The combined organic layer was concentrated under vacuum and isopropyl alcohol was added to the obtained residue. The reaction mixture was concentrated till 5 volumes remain. Hydrochloric acid in isopropyl ether (15 g) was added to the reaction mixture which was stirred for about 12 h to about 16 h. The solid obtained was filtered, washed with isopropyl alcohol and dried under vacuum at about 45° C. to about 55° C. Yield: 12 g

### Example 7

A mixture of daclatasvir dihydrochloride (50 g) in purified water (250 mL) was stirred to give a clear solution. The solution was filtered and the filtrate was heated to about 90° C. to about 100° C. The reaction mixture was stirred for about 4 h to about 5 h. The reaction mixture was cooled to about 20° C. to about 30° C. and stirred for about 12 h to about 15 h. The solid obtained was filtered, washed with water and dried under following conditions—
a. about 40° C. to about 50° C. in vacuum tray dryer for about 12 h
b. about 30° C. to about 35° C. in air oven for about 12 h
c. about room temperature on rotavapor under vacuum for about 12 h
Yield: 75-90 g
XRD peaks of daclatasvir dihydrochloride hydrate under drying conditions (a):

| Pos. [°2Th.] | d-spacing [Å] | Rel. Int. [%] |
|---|---|---|
| 8.23 | 10.73 | 42.95 |
| 9.51 | 9.29 | 100.00 |
| 11.03 | 8.01 | 10.06 |
| 13.17 | 6.72 | 11.42 |
| 13.58 | 6.52 | 23.37 |
| 14.88 | 5.95 | 24.10 |
| 15.09 | 5.86 | 14.81 |
| 15.31 | 5.78 | 52.79 |
| 18.97 | 4.67 | 79.95 |
| 19.15 | 4.63 | 10.93 |
| 21.02 | 4.22 | 18.65 |
| 21.24 | 4.18 | 14.83 |
| 21.51 | 4.13 | 20.33 |
| 22.22 | 4.00 | 30.50 |
| 22.50 | 3.95 | 21.37 |
| 23.20 | 3.83 | 32.54 |
| 24.13 | 3.68 | 17.98 |
| 24.26 | 3.66 | 15.11 |
| 24.70 | 3.60 | 58.43 |
| 25.31 | 3.51 | 26.44 |
| 28.66 | 3.11 | 10.16 |
| 30.53 | 2.92 | 11.28 |

TGA analysis of daclatasvir dihydrochloride hydrate under drying conditions (a) shows a weight loss of about 7.5 weight % up to 100° C. determined over the temperature range of 0° C. to 350° C. and heating rate 10° C./min.
DSC analysis of daclatasvir dihydrochloride hydrate under drying conditions (a) shows endothermic peak at about 227° C.
Water content of daclatasvir dihydrochloride hydrate under drying conditions (a) by KF method: 7.56%
XRD peaks of daclatasvir dihydrochloride hydrate under drying conditions (b):

| Pos. [°2Th.] | d-spacing [Å] | Rel. Int. [%] |
|---|---|---|
| 8.24 | 10.72 | 36.95 |
| 9.52 | 9.28 | 100.00 |
| 11.04 | 8.01 | 9.34 |
| 13.18 | 6.71 | 12.24 |
| 13.58 | 6.51 | 21.10 |
| 14.88 | 5.94 | 24.66 |
| 15.10 | 5.86 | 12.93 |
| 15.31 | 5.78 | 52.26 |
| 18.98 | 4.67 | 80.63 |
| 19.16 | 4.63 | 10.56 |
| 21.03 | 4.22 | 18.94 |
| 21.25 | 4.18 | 14.49 |
| 21.53 | 4.12 | 20.95 |
| 22.23 | 3.99 | 30.15 |
| 22.52 | 3.94 | 19.50 |
| 23.21 | 3.83 | 34.04 |
| 24.14 | 3.68 | 18.63 |
| 24.28 | 3.66 | 16.70 |
| 24.72 | 3.60 | 58.15 |
| 25.32 | 3.51 | 25.07 |
| 28.67 | 3.11 | 9.04 |
| 30.54 | 2.92 | 10.71 |

TGA analysis of daclatasvir dihydrochloride hydrate under drying conditions (b) shows a weight loss of about 7.5 weight % up to 100° C. determined over the temperature range of 0° C. to 350° C. and heating rate 10° C./min.
DSC analysis of daclatasvir dihydrochloride hydrate under drying conditions (b) shows endothermic peak at about 227° C.
Water content of daclatasvir dihydrochloride hydrate under drying conditions (b) by KF method: 9.42%
XRD peaks of daclatasvir dihydrochloride hydrate under drying conditions (c):

| Pos. [°2Th.] | d-spacing [Å] | Rel. Int. [%] |
|---|---|---|
| 8.27 | 10.69 | 36.27 |
| 9.55 | 9.26 | 100.00 |
| 11.06 | 7.99 | 9.51 |
| 13.20 | 6.70 | 12.05 |
| 13.61 | 6.50 | 20.87 |
| 14.92 | 5.93 | 24.46 |
| 15.12 | 5.85 | 13.70 |
| 15.34 | 5.77 | 52.57 |
| 19.01 | 4.66 | 80.60 |
| 19.19 | 4.62 | 11.23 |
| 21.06 | 4.21 | 18.06 |
| 21.27 | 4.17 | 14.88 |
| 21.55 | 4.12 | 21.38 |
| 22.26 | 3.99 | 29.30 |
| 22.54 | 3.94 | 20.73 |
| 23.24 | 3.82 | 33.28 |
| 24.17 | 3.68 | 18.67 |
| 24.31 | 3.66 | 16.39 |
| 24.74 | 3.59 | 55.54 |
| 25.35 | 3.51 | 24.75 |
| 28.71 | 3.10 | 9.16 |
| 30.57 | 2.92 | 10.96 |

TGA analysis of daclatasvir dihydrochlotide hydrate under drying conditions (c) shows a weight loss of about 7.7 weight % up to 100° C. determined over the temperature range of 0° C. to 350° C. and heating rate 10° C./min.
DSC analysis of daclatasvir dihydrochloride hydrate under drying conditions (c) shows endothermic peak at about 225° C.
Water content of daclatasvir dihydrochloride hydrate under drying conditions (c) by KF method: 7.96%

### EXAMPLE 8

### Preparation of Amorphous Daclatasvir Dihydrochloride

(A) To a stirred mixture of daclatasvir (2 g) in acetone (30 mL) was added hydrochloric acid in isopropyl ether (1.6 g). The reaction mixture was stirred for about 2 h to about 3 h. The precipitated solid was filtered under nitrogen, washed with acetone and dried under vacuum at about 40° C. to about 45° C.
(B) To a stirred mixture of daclatasvir (2 g) in acetonitrile (20 mL) was added hydrochloric acid in isopropyl ether (1.6 g). The reaction mixture was stirred for about 2 h to about 3 h. The precipitated solid was filtered under nitrogen, washed with acetonitrile and dried under vacuum at about 40° C. to about 45° C.
(C) To a stirred mixture of daclatasvir (2 g) in toluene (20 mL) was added hydrochloric acid in isopropyl ether (1.6 g). The reaction mixture was stirred for about 2 h to about 3 h. The precipitated solid was filtered under nitrogen, washed with toluene and dried under vacuum at about 40° C. to about 45° C.
(D) A mixture of daclatasvir dihydrochloride (2 g) in water (8 mL) was stirred to give a clear solution and acetone (3 mL) was added to it. The reaction mixture was concentrated under vacuum at about 60° C. to about 65° C. to give amorphous daclatasvir dihydrochloride.
(E) A mixture of daclatasvir dihydrochloride (2 g) in water (8 mL) was stirred to give a clear solution and methanol (3 mL) was added to it. The reaction mixture was concentrated under vacuum at about 60° C. to about 65° C. to give amorphous daclatasvir dihydrochloride.
(F) A mixture of daclatasvir dihydrochloride (2 g) in water (8 mL) was stirred to give a clear solution and tetrahydrofuran (3 mL) was added to it. The reaction mixture was concentrated under vacuum at about 60° C. to about 65° C. to give amorphous daclatasvir dihydrochloride.
(G) A mixture of daclatasvir dihydrochloride (2 g) in water (8 mL) was stirred to give a clear solution and acetonitrile (3 mL) was added to it. The reaction mixture was concentrated under vacuum at about 60° C. to about 65° C. to give amorphous daclatasvir dihydrochloride.
(H) A mixture of daclatasvir dihydrochloride (2 g) in water (8 mL) was stirred to give a clear solution and isopropyl alcohol (3 mL) was added to it. The reaction mixture was concentrated under vacuum at about 60° C. to about 65° C. to give amorphous daclatasvir dihydrochloride.
(I) A mixture of daclatasvir dihydrochloride (10 g) in methanol (100 mL) was stirred to give a clear solution. The solution obtained was spray dried using spray dryer with following conditions to give amorphous daclatasvir dihydrochloride.
a. Feed Rate 25%
(ii) Aspirator 1800 RPM
(iii) Inlet temperature 65-70° C.
(iv) Pressure 2 Kg
(J) A mixture of daclatasvir dihydrochloride (5 g) in purified water (30 mL) was stirred to give a clear solution and charcoal was added to it. The reaction mixture was filtered through Hyflo bed and was washed with purified water. The filtrate was charged into a flask and the solution freeze by placing in liquid nitrogen bath. The freeze solution was lyophilized for about 12 h to about 16 h to give amorphous daclatasvir dihydrochloride.

### EXAMPLE 9

### Preparation of Daclatasvir Dihydrochloride hydrate

To a stirred mixture of daclatasvir (5 g) in water (50 mL) was added concentrated hydrochloride acid (2.8 g). The reaction mixture was heated to about 90° C. to about 100° C. and stirred for about 7 h. The reaction mixture was cooled to about room temperature and stirred for about 16 h. The solid obtained was filtered, washed with water and dried in vacuum tray dryer at about 30° C. to about 35° C. for about 16 h to about 20 h. Yield: 9.3 g Water content by KF method: 8.68%

### EXAMPLE 10

### Preparation of 1-pyrrolidinecarboxylic acid, 2,2′-([1,1′-biphenyl]-4,4′-diyldi-1H-imidazole-5,2-diyl)bis-1,1′-bis(1,1-dimethylethyl)ester, (2S,2′S)—

4,4′-Bis(2-bromoacetyl)biphenyl 100 g was added to methylene dichloride (1000 ml) and stirred to get solution. Boc-L-Proline (120 g) was added to it. Diisopropylethylamine 68.5 g was added to the reaction mass at 15-25° C. The reaction mass was stirred for about 4 to 5 h. The reaction mass was quenched by adding water. The aqueous layer was separated and organic layer was washed with aqueous acetic acid solution (Cone HCl also can be used instead of acetic acid) till pH of aqueous layer was between 4-6. The organic layer was washed with water and distilled under vacuum to get a residue. The residue was dissolved in toluene and toluene solution was used further. Ammonium acetate 390 g was added to the toluene solution and the reaction mass was stirred. The reaction mass heated to 95-105° C. and maintained till completion of reaction. After completion of the reaction, the reaction mass was cooled to 50-60° C., methanol was added and the reaction mass cooled to 20-30° C. and stirred for 2 to 3 hours. The solid was filtered and washed with toluene. Purity of crude:—93.92%. Crude wet cake was stirred in mixture of toluene, methanol and acetic acid and the reaction mass was heated to 60-70° C., water was added to reaction mass at 60-70° C. and the reaction mass cooled to 20-30° C. and stirred. The solid obtained was filtered and washed with toluene and then with water. Wet solid was dried. Purity—97.04%. The above purification was repeated to obtain 60.0 g of the title compound in a purity of 98.97%.

### EXAMPLE 11

### Preparation of 5,5′-(4,4′-biphenyldiyl)bis(2-((2S)-2-pyrrolidinyl)-1H-imidazole)tetrahydrochloride

60 g of compound obtained as per example 10 was added into isopropyl alcohol 180 ml. The reaction mass was stirred. Water was added followed by addition of concentrated aqueous hydrochloride 90 ml. The reaction mass was heated to 50-55° C. and maintained till completion of reaction. After completion of reaction (3-5 h), isopropyl alcohol was added. The reaction mass was cooled to 20-30° C. and stirred for 2 to 3 hours. The solid obtained was filtered and washed with 60 ml isopropyl alcohol. Purity—99.28%. The wet cake was added into methanol and heated to 50-55° C. under stirring. The reaction mass was maintained for 1 hour at 50-55° C. and cooled to 20-30° C. and stirred for 2 to 3 hours. The solid obtained was filtered and washed with 60 ml methanol. Solid dried in VTD. Purity:—99.86%.

### EXAMPLE 12

### Preparation of Daclatasvir Dihydrochloride Hydrate

Moc-L-Valine 76.8 g was added into 1000 ml acetonitrile. The reaction mass was stirred and 59.2 g HOBt and 80.7 g of EDC.HCl were added and the reaction mass stirred for about 1 hour. The reaction mass was cooled to 15-20° C. and 100 g of compound obtained from example 11 was added. 90.6 g of diisopropylethylamine was added to the reaction mass at 0-10° C. After complete addition, the reaction mass was maintained at 15-20° C. After completion of reaction, water and methylene dichloride were added. The reaction mass was stirred and aqueous and organic layers were separated. The aqueous layer was extracted with methylene dichloride and the combined organic layers were washed with sodium hydroxide solution and then once with 10% acetic acid solution and twice with 5% acetic acid solution followed by washing with sodium bicarbonate solution. The organic layer was distilled under vacuum and the residue dissolved in acetone and methanol mixture. 54.7 g of malonic acid was added to solution and heated to 40-45° C. and stirred for about 1 hours. The reaction mass was cooled to 20-25° C. and stirred for 12 hours. The malonate salt was filtered and washed with acetone. The wet cake was crystallized using acetone. The solid was filtered and washed with acetone (purity 99.75%). Wet cake was dissolved in water and ethyl acetate was added. The reaction mass was basified using aqueous ammonia solution, pH not less than 8.5. The aqueous and organic layers were separated. The organic layer was stirred with water and concentrated HCl added till pH of aqueous layer was in between 1-2. Aqueous layer was separated and charcoaled. Solution filtered through hyflo and hyflo was washed with water. About 50 ml water was distilled from filtrate under vacuum and the reaction mass was heated to 50-55° C. and stirred for 2-3 hours. The reaction mass was cooled to 20-30° C. and then stirred for 12 hours. The solid obtained was filtered and washed with water and dried in air oven at 50-60° C. Isolated dry weight. 70.0 g. Purity 99.86%. KF value:—˜8.4%

### EXAMPLE 13

### Preparation of Daclatasvir Dihydrochloride Hydrate

100 g of Daclatasvir dihydrochloride hydrate obtained as per example 12, was stirred in 500 ml water and 1000 ml of methylene dichloride. The reaction mass was basified using aqueous ammonia solution, pH not less than 8.5. The aqueous and organic layers were separated and washed with 5% sodium hydroxide solution containing 5% sodium chloride then with 5% sodium chloride solution and once with 5% acetic acid solution. Finally organic layer washed with water. Organic layer was stirred with water 400 ml and concentrated HCl added till pH of aqueous layer was in between 1-2. Aqueous layer was separated and filtered through micron filter paper. About 50 ml water was distilled from filtrate under vacuum and then reaction mass was heated to 50-55° C. and stirred for 2-3 hours. The reaction mass was cooled to 20-30° C. and stirred for 12 hours. The solid Obtained was filtered and washed with water and dried in air oven at 50-60°. Isolated dry weight 75.0 g. Purity 99.9%. KF ˜7.8%

### EXAMPLE 14

### Preparation of Daclatasvir Dihydrochloride Hydrate

100 g of Daclatasvir dihydrochloride hydrate obtained as per example 12, was stirred in water 500 ml and ethyl acetate 1000 ml. The reaction mass was basified using aqueous ammonia solution, pH not less than 8.5. Aqueous and organic layers were separated. Organic layer was stirred with water 400 ml and concentrated HCl added till pH of aqueous layer was in between 1-2. Aqueous layer was separated and filtered through micron filter paper. About 50 ml water was distilled from filtrate under vacuum and the reaction mass was heated to 50-55° C. and stirred for 2-3 hours. During this period solid starts to precipitate. The reaction mass was cooled to 20-30° C. and then stirred for 12 hours. The solid obtained was filtered and washed with water and dried in air oven at 50-60° C. KF ˜8.4%

1. Crystalline daclatasvir dihydrochloride hydrate wherein the molar ratio of daclatasvir dihydrochloride and water is in the range of 1:0.5 to 1:6.

2. The crystalline daclatasvir dihydrochloride hydrate of claim 1, wherein the molar ratio of daclatasvir dihydrochloride and water is in the range of 1:3 to 1:5.

3. The crystalline daclatasvir dihydrochloride hydrate of claim 1, characterized by an X-ray powder diffraction (XRPD) spectrum having peak reflections at about 9.5, 11.0, 23.2 and 24.3±0.2 degrees 2 theta.

4. The crystalline daclatasvir dihydrochloride hydrate of claim 3, further characterized by an XRPD spectrum having peak reflections at about 8.3 and 30.6±0.2 degrees 2 theta.

5. The crystalline daclatasvir dihydrochloride hydrate of claim 1, characterized by a TGA thermogram, showing a weight loss of about 1 weight % to 13 weight % up to 100° C. determined over a temperature range of 0° C. to 350° C. and a heating rate of 10° C./min.

6. The crystalline daclatasvir dihydrochloride hydrate of claim 1, characterized by DSC thermogram having endothermic peak at about 226±4° C.

7. The crystalline daclatasvir dihydrochloride hydrate of claim 1, characterized by data selected from the group consisting of: an XRPD pattern as depicted in FIG. 1, a TGA thermogram as depicted in FIG. 2; a DSC thermogram as depicted in FIG. 3; and any combination thereof.

8. (canceled)

9. (canceled)

10. (canceled)

11. (canceled)

12. A process for the preparation of crystalline daclatasvir dihydrochloride hydrate, the process comprising:
(a) dissolving daclatasvir dihydrochloride in water, optionally in the presence of a solvent, to form a solution;
(b) heating the solution obtained in step (a) to a temperature of about 40° C. to 110° C.;
(c) optionally maintaining the solution of step (b) at the temperature of about 40° C. to 110° C.;
(d) cooling the solution of step (b) or step (c) to a temperature of about −5° C. to 35° C.;
(e) isolating the crystalline daclatasvir dihydrochloride hydrate; and
(f) drying the isolated crystalline daclatasvir dihydrochloride hydrate of step (e) at a temperature in the range of about 25° C. to about 60° C.

13. (canceled)

14. A salt of daclatsavir with malonic acid, characterized by a proton NMR spectrum having peak positions at 11.79 (br, 2H), 7.78-7.80 (dd, 4H), 7.69-7.71 (dd, 4H), 7.55 (s, 2H), 7.31-7.33 (d, 2H), 5.08-5.10 (t, 2H), 4.08 (t, 2H), 3.8 (m, 4H) 3.54 (s, 6H), 3.2 (s, 4H), 1.95-2.17 (m, 10), 0.79-0.91 (d, 6H), 0.84-0.86 (d, 6H).

15. (canceled)

16. The process for the preparation of daclatasvir dihydrochloride of claim 12, further comprising:
(a) reacting daclatasvir with malonic acid to form daclatasvir malonate;
(b) reacting daclatasvir malonate with a base to form daclatasvir;
(c) reacting the daclatasvir of step b with hydrochloric acid to form daclatasvir dihydrochloride.

17. (canceled)

18. (canceled)
